(19) United States
(12) Patent Application Publication
(10) Pub. No.: US 2011/0060345 A1
(43) Pub. Date: Mar. 10, 2011

(54) SURGICAL ROBOT SYSTEM AND EXTERNAL FORCE MEASURING METHOD THEREOF

(76) Inventors: Min Cheol Lee, Busan (KR); Seung Wook Choi, Seongnam-si (KR)

(21) Appl. No.: 12/797,867

(22) Filed: Jun. 10, 2010

(30) Foreign Application Priority Data
Sep. 9, 2009 (KR) 10-2009-0084720

(51) Int. Cl. A61B 19/00

(52) U.S. Cl. 606/130

(57) ABSTRACT

A surgical robot system and an external force measuring method of the surgical robot system are disclosed. The surgical robot system, which includes: a driving motor unit configured to generate and output an encoder signal corresponding to state information of a system; and a controller unit configured to receive the encoder signal as input and compute an external force applied on an instrument using an SMCSPO (sliding mode control with sliding perturbation observer) algorithm, can obtain information on the operational force of the instrument by an indirect method, making it possible to implement a technology for a realistic sensory device.

### CROSS-REFERENCE TO RELATED APPLICATIONS

This application claims the benefit of Korean Patent Application No. 10-2009-0084720 filed with the Korean Intellectual Property Office on Sep. 9, 2009, the disclosures of which are incorporated herein by reference in their entirety.

### BACKGROUND

The present invention relates to a surgical robot system, more particularly to a surgical robot system and an external force measuring method thereof.

A surgical robot system refers to a robot system capable of performing surgical procedures which were hitherto performed by surgeons. The surgical robot can provide more accurate and precise operations compared to a human, and also enables remote surgery.

Generally, when performing surgery using a surgical robot system, a surgeon may manipulate a master robot to control the movement of a surgical instrument from a surgery location that is away from the patient (for example, a different room from the one occupied by the patient). The master robot may generally include one or more manual input devices, such as handheld wrist gimbals, joysticks, exoskeletal gloves, handpieces, etc. The operation of a driving motor unit coupled to a controller unit may be controlled by the manipulation of the surgeon using the manual input device, whereby the control for the position, direction, and action of the instrument may be provided. That is, the driving motor unit may control the instrument, which is directly inserted into the opened surgical site, to perform various actions involved in surgical procedures (for example, incising a tissue, grasping a blood vessel, etc.).

Since, with a surgical robot system, the surgery is generally performed on a patient by a surgeon's manipulation from a remote location, there is a need to provide information to the surgeon regarding the operational force caused by the instrument.

It can be said that the information regarding the operational force of the instrument relates to the forces and torques applied on the end portion of the instrument. However, due to the nature of the instrument, which is inserted into a patient's body to conduct surgery, sensors for measuring the operational force cannot be attached to the instrument.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An objective of the invention is to provide a surgical robot system and an external force measuring method of the surgical robot system, with which the operational force of the instrument can be measured by an indirect method.

Another objective of the invention is to provide a surgical robot system and an external force measuring method of the surgical robot system, which can implement a technology for a realistic sensory device by providing information on the operational force of an instrument obtained by an indirect method.

Also, an objective of the invention is to provide a surgical robot system and an external force measuring method of the surgical robot system, which can implement a technology for a realistic sensory device and thereby make it possible to perform surgery more safely.

Another objective of the invention is to provide a surgical robot system and an external force measuring method of the surgical robot system, which by measuring the operational force of the instrument and adjusting the strength accordingly, can avoid damaging a patient's internal organ while holding the organ during surgery, and which make it possible to conduct surgery safely.

Additional objectives of the invention will be apparent from the written description below.

One aspect of the invention provides a surgical robot system that includes: a driving motor unit configured to generate and output an encoder signal corresponding to state information of a system; and a controller unit configured to receive the encoder signal as input and compute an external force applied on an instrument using an SMCSPO (sliding mode control with sliding perturbation observer) algorithm.

The encoder signal can include information regarding one or more of a rotation angle of a motor and a rotation angular velocity of a motor.

The controller unit using the SMCSPO algorithm can include: a sliding state observer configured to estimate a state variable by using the state information of the system; and a perturbation observer configured to compute a perturbation value by using the estimated state variable.

The perturbation observer can compute the perturbation value using the following equation, in which \hat{ψ}ⱼ is the perturbation value, and χ₃ⱼ is gain.

\hat{ψ}_{J}=α_{3J}(−\hat{x}_{3J}|α_{3J}\hat{x}₂ⱼ)

Another aspect of the invention provides a method of measuring an external force applied on an effector of a surgical robot system, which includes a driving motor unit, an instrument, and a controller unit, where the method includes: receiving as input an encoder signal, which corresponds to state information of a system; and computing the external force applied on the effector by using the inputted encoder signal and an SMCSPO (sliding mode control with sliding perturbation observer) algorithm.

The encoder signal can include information regarding one or more of a rotation angle of a motor and a rotation angular velocity of a motor.

A state variable corresponding to the state information of the system can be estimated by way of the SMCSPO algorithm, and the estimated state variable can be used to compute a perturbation value, which represents the external force.

The perturbation value can be computed using the following equation, in which \hat{ψ}ᵢ is the perturbation value, and α₃ᵢ is gain.

\hat{ψ}_{J}=α_{3J}(−\hat{x}_{3J}|α_{3J}\hat{x}_{zj})

Additional aspects, features, and advantages, other than those described above, will be obvious from the drawings, claims, and written description below.

### BRIEF DESCRIPTION OF THE DRAWINGS

FIG. 1 is a diagram schematically illustrating the structure of a surgical robot system according to an embodiment of the invention.

FIG. 2 is a flow diagram illustrating the operation of a controller unit according to an embodiment of the invention.

FIG. 3 is a diagram illustrating the schematics of an SMCSPO (sliding mode control with sliding perturbation observer) algorithm according to an embodiment of the invention.

### DETAILED DESCRIPTION

As the invention allows for various changes and numerous embodiments, particular embodiments will be illustrated in the drawings and described in detail in the written description. However, this is not intended to limit the invention to particular modes of practice, and it is to be appreciated that all changes, equivalents, and substitutes that do not depart from the spirit and technical scope of the present invention are encompassed in the invention. In the written description, certain detailed explanations of related art are omitted when it is deemed that they may unnecessarily obscure the essence of the invention.

While such terms as “first” and “second,” etc., may be used to describe various components, such components must not be limited to the above terms. The above terms are used only to distinguish one component from another.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Certain embodiments of the invention will be described below in detail with reference to the accompanying drawings. Those components that are the same or are in correspondence are rendered the same reference numeral regardless of the figure number, and redundant descriptions are omitted.

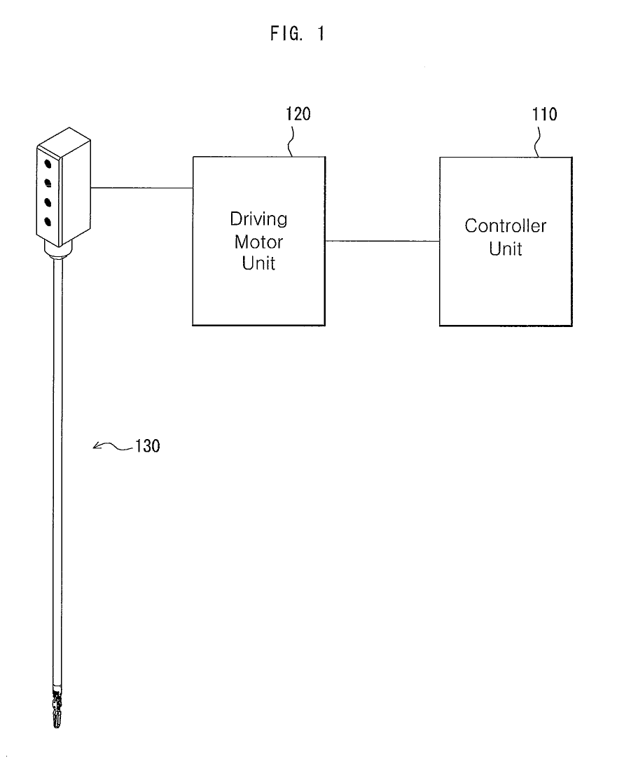

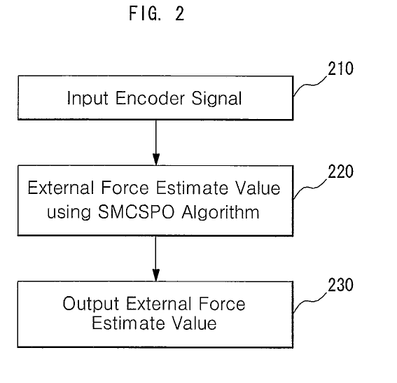

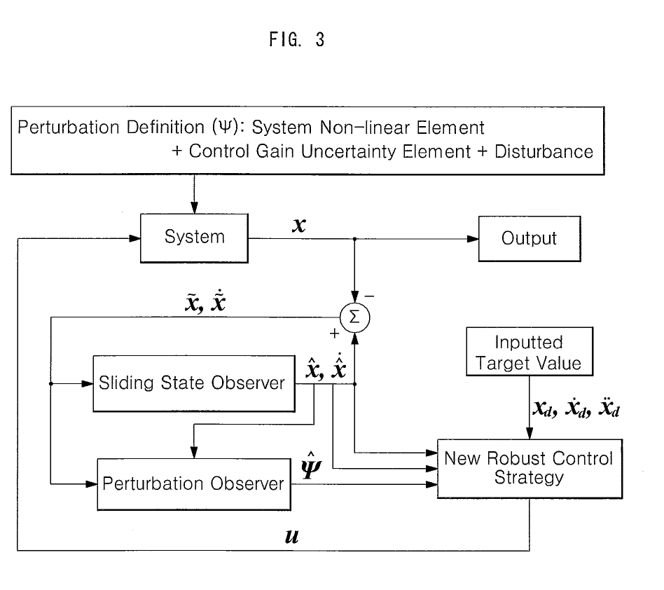

FIG. 1 is a diagram schematically illustrating the structure of a surgical robot system according to an embodiment of the invention, FIG. 2 is a flow diagram illustrating the operation of a controller unit according to an embodiment of the invention, and FIG. 3 is a diagram illustrating the schematics of an SMCSPO (sliding mode control with sliding perturbation observer) algorithm according to an embodiment of the invention.

Referring to FIG. 1, a surgical robot system may include a controller unit 110, a driving motor unit 120, and an instrument 130.

The controller unit 110 may control the driving motor unit 120 to operate in correspondence to the manipulation of the surgeon on a manual input device equipped on the master robot. The manual input device can include, for example, a handheld wrist gimbal, a joystick, an exoskeletal glove, a handpiece, etc.

Also, the controller unit 110 may be equipped with an observer. The observer can approximate an external force applied on the effector of the instrument 130 by using the SMCSPO (sliding mode control with sliding perturbation observer) algorithm, which is used for improving the manipulation performance of a non-linear system. In calculating the external force applied on the effector, the observer of the controller unit 110 can use an encoder signal inputted from an encoder included in the driving motor unit 120. This will be described later in further detail with reference to the relevant drawings.

The driving motor unit 120 may include a motor, which may rotate in a direction and/or number of revolutions corresponding to a control signal inputted from the controller unit 110, and an encoder, which may compute the information on the revolutions and/or angular velocity, etc., of the motor and provide it to the controller unit 110. The motor can be, for example, a servomotor.

The driving motor unit 120 can also further include a motor driving circuit for rotating the motor in a direction and/or number of revolutions corresponding to a control signal inputted from the controller unit 110.

In one example, the driving motor unit 120 can be coupled to a pulley included in the instrument 130, and can manipulate the effector, which may be connected to the pulley by a wire, in a manner corresponding to the rotation direction and number of revolutions of the motor.

A description will now be provided, with reference to FIG. 2 and FIG. 3, on a method of calculating an external force applied on the effector by using an encoder signal.

Referring to FIG. 2, in operation 210, the observer of the controller unit 110 may receive an encoder signal as input from the encoder of the driving motor unit 120. The encoder signal can include, for example, information regarding one or more of current angle, current angular velocity, rotation angle, rotation angular velocity, etc.

In operations 220 and 230, the observer may calculate and output the external force applied on the effector, using the SMCSPO (sliding mode control with sliding perturbation observer) algorithm.

In general, the equation of motion for a second order system having n degrees of freedom can be expressed by Equation 1 as follows.

$${\overline{x}}_{j} = {f}_{j} ( z ) + Δ {f}_{j} ( z ) + {\sum }_{i = 1}^{n} [ ( {b}_{ji} ( z ) + Δ {b}_{ji} ( z ) ) {u}_{1} ] + {d}_{j} ( t ) \qquad [ Equation 1 ]$$

Here, z is a state vector and can be expressed as z≡[Z₁, . . . , Zₙ]^{T}, while Z₁ is a state variable and can be expressed as Zⱼ≡[xⱼx*]. Δfⱼ(z) represents non-linear elements and uncertainty, and Δbⱼᵢ(z) represents uncertainty in the control gain matrix element. dⱼ represents disturbance, uᵢ represents control input, and fⱼ(z) and bⱼᵢ(z) represent continuous state functions, respectively. Here, i is to denote an element of the control gain matrix that is influenced by each of the control inputs.

As illustrated in FIG. 3, perturbation may be defined by the non-linear elements, uncertainty, and disturbance, etc., in the equation of motion in Equation 1 and can be expressed by Equation 2 as follows.

$${ψ}_{j} ( z , t ) = Δ {f}_{j} ( z ) + {\sum }_{i = 1}^{n} [ Δ {b}_{ji} ( z ) {u}_{1} ] + {d}_{j} ( t ) \qquad [ Equation 2 ]$$

If it is assumed that the terms defining perturbation are smaller than certain known continuous functions, then the following Equation 3 can be obtained.

$${Γ}_{j} ( z , t ) = {F}_{j} ( z ) + {\sum }_{i = 1}^{n} \vert {Φ}_{ji} ( z ) {u}_{i} \vert + {D}_{j} ( t ) > \vert {Ψ}_{j} ( t ) \vert \qquad [ Equation 3 ]$$

Here, Fⱼ(z)>|Δfⱼ|, φⱼᵢ>|Δbⱼᵢ|, and Dⱼ>|dⱼ|, such that each perturbation component has an upper bound.

The sliding state observer may serve to observe the state variables, and the perturbation observer may serve to compensate the control input for the perturbation caused by system uncertainty. The sliding state observer may be configured to be capable of observing state variables with quick response characteristics, and the perturbation observer may be configured to be capable of estimating the perturbation term, which is a non-linear component, with a quick response.

The equation of motion provided for the sliding state observer can be expressed by state space representation as Equation 4 below.

$${\dot{x}}_{1 j} = {x}_{2 j} \\ {\dot{x}}_{2 j} = {f}_{j} ( z ) + {\sum }_{i = 1}^{n} {b}_{ji} ( z ) {u}_{1} + {Ψ}_{j} \\ y = {x}_{1 j} \qquad [ Equation 4 ]$$

Here, if it is assumed that the only measurable information is position information, then the observers may, in spite of the uncertain elements, perform the task of estimating those state vectors that cannot be measured. The following Equation 5 mathematically represents the structure of the sliding state observer.

[Equation 5]

{circumflex over (\dot{x}₁ⱼ=\hat{x}₂ⱼ−k₁ⱼsat(\tilde{x}₁ⱼ)−α₁ⱼ\tilde{x}₁ⱼ

{circumflex over (\dot{x}₂ⱼ=α₃ūⱼ−k₂ⱼsat(\tilde{x}₁ⱼ)−α₂ᵢ x̅₁ⱼ−S₀ⱼ+\hat{ψ}ⱼ

Here, k₁ⱼ, k₂ⱼ, α₁ⱼ, α₂ⱼ, which have positive values, are gains of the observers, while \tilde{x}₁ⱼ=\hat{x}₁ⱼ−x₁₁, representing estimate errors of the state variables, and S₀ⱼ=\tilde{x}₁ⱼ+rⱼ\tilde{x}₂ⱼ represents a sliding plane formed by the estimate errors. The symbol “̂” represents a result estimated by an observer. By subtracting Equation 4 from Equation 5, the error equations of motion of the observer can be computed as Equation 6 below.

[Equation 6]

{tilde over (\dot{x}=\tilde{x}₂ⱼ−k₁ⱼsat(\tilde{x}ᵢⱼ)−α₁ⱼ\tilde{x}₂j

{circumflex over (\dot{x}₂ⱼ=−k₂ⱼsat(\tilde{x}₁ⱼ)−α₂ⱼ\tilde{x}₁ⱼ−s₀₁−ψ₁

Here, assuming that \tilde{f}=f(\hat{z}) is included in Δf and that \tilde{b}=b(\hat{z})−b(z) is included in Δb, \tilde{ψ} can be referred to as perturbation as defined by Equation 2. Since the sign of \tilde{x}₁ⱼ changes discontinuously, a saturation function can be used, so that k₁ⱼ, k₂ⱼ may change continuously when they are within ε^{0j}, which is the boundary of the sliding state observer. The saturation function (sat(\tilde{x}₁ⱼ)) may be defined by Equation 7 as follows.

$$sat ( {\overline{x}}_{1 j} ) - \{ \begin{matrix}\frac{{\hat{x}}_{1 j}}{\vert {\hat{x}}_{1 j} \vert} , & if \vert {\hat{x}}_{1 j} \vert \geq {ɛ}_{0 j} \\ \frac{{\tilde{x}}_{1 j}}{{ɛ}_{0 j}} , & if \vert {\tilde{x}}_{1 j} \vert < {ɛ}_{0 j} \end{matrix} \qquad [ Equation 7 ]$$

The sliding surface of the sliding observer may be composed of \tilde{x}₁ⱼ, \hat{x}₂ⱼ, and a sliding mode may be obtained along the line \hat{x}₁ⱼ=0. When \tilde{x}₂ⱼ is made to satisfy 0 according to the sign of the \tilde{x}₁ⱼ, then \tilde{x}₂ⱼ may follow the state space locus shown in Equation 8.

[Equation 8]

\tilde{x}₂ⱼ≧α₁\tilde{x}₁ⱼ(\tilde{x}₁ⱼ>0)

\tilde{x}₂ⱼ>α₁\tilde{x}₁ⱼ−k₁j

When there is a sliding mode in an observer, the error equation of motion of Equation 6 described above may take the form of a filter which is inputted with perturbation having a cut-off frequency of

$$\frac{{k}_{2 j}}{{k}_{1 j}}$$

and which outputs \tilde{x}₂ⱼ.

In determining the stability of the sliding state observer, if k₂ⱼ≧Γ(\hat{z},t) is satisfied, then |\tilde{x}₂ⱼ|≦k₁ⱼ is satisfied in Equation 8. Thus, \tilde{x}₂ⱼ has an upper bound of k₁ⱼ, guaranteeing stability. That is, since Γ(\hat{z},t) has an upper bound of ψⱼ, the uncertainty of the observer is negligible, compared to the uncertainty of the mathematical modeling and external disturbances. Therefore, it can be seen that the observer error is decreased according to an increase of the cut-off frequency regardless of disturbance, and while k₂ⱼ can be selected as a value higher than the upper bound of the perturbation, the lower bound of k₂ⱼ may be selected, considering the problem of chatter.

By having the sliding state observer estimate the state variables required by the perturbation observer, and having the perturbation observer estimate the non-linear components of the parallel manipulator, disturbance, uncertainty, etc., to be utilized in the control, it is possible to implement a very powerful controller.

Before coupling the sliding state observer to the sliding mode controller, a couple of control variables from the equations of motion may be separated as in Equation 9 below.

$${f}_{j} ( \hat{x} ) + {\sum }_{i = 1}^{u} {b}_{ji} ( \hat{x} ) {u}_{i} = {α}_{3 j} {\overline{u}}_{j} \qquad [ Equation 9 ]$$

Here, αₐᵢ is a constant having a positive value, and ūᵢ is a newly defined control variable. Thus, the control input can be expressed as Equation 10 below.

[Equation 10]

uⱼ=B⁻¹Col[α₃ⱼūⱼ−fⱼ(\hat{z})]

Here, since B is [bⱼᵢ(\hat{z})]ₙₓₛ, the equations of motion can be simplified by the definition in Equation 10 as Equation 11.

[Equation 11]

\dot{x}₁ⱼ=x₂ⱼ

\dot{x}₂ⱼ=α₃ⱼūⱼ+ψ₁

yⱼ=x₁ⱼ

Similarly, the structure of the sliding state observer can also be simplified as Equation 12 below.

[Equation 12]

{circumflex over (\dot{x}₁ⱼ=\hat{x}₂ⱼ−k₁ⱼsat(\tilde{x}₁ⱼ)α₁ⱼ\tilde{x}₁j

{circumflex over (\dot{x}₂ⱼ=α₃ūⱼ−k₂ⱼsat(\hat{x}₁ⱼ)−α₂ⱼ\tilde{x}₁ⱼ−s₀ⱼ+\hat{ψ}ⱼ

In order that the perturbation observer according to this embodiment may calculate the perturbation without the attachment of additional sensors, a new state variable x₃ⱼ is defined, so that the perturbation can be calculated by the other variables as in the following Equation 13.

$${x}_{3 j} = {α}_{3 j} {x}_{2 j} - \frac{{Ψ}_{j}}{{α}_{3 j}} \qquad [ Equation 13 ]$$

Here, it is assumed that \dot{ψ}ⱼ exists in the form of a continuous function and that the spectrum of ψⱼ exists within a known finite frequency band. By finding a first derivative of Equation 13, the following Equation 14 can be obtained.

$${\dot{x}}_{3 j} = {α}_{3 j} {\dot{x}}_{2 j} - \frac{{Ψ}_{j}}{{α}_{3 j}} \qquad [ Equation 14 ]$$

If α₃ⱼ is increased to a level that renders the effect of \dot{ψ}ⱼ negligible in Equation 14, then x₃ⱼ can be observed well in spite of the effect of perturbation. Using this, a perturbation observer model capable of observing ψⱼ and x₃ⱼ may be deduced, as shown in Equation 15 below, and coupled with the sliding state observer.

[Equation 15]

{circumflex over (\dot{x}₃ⱼ=α₃j³(−\hat{x}₃ⱼ+α₃ⱼx₂ⱼ+ūⱼ)

\hat{ψ}ⱼ=αₐⱼ(−\hat{x}ₐⱼ+αₐⱼx₃ⱼ)

By taking the difference between Equations 15 and 14 and substituting ψᵢ as worked out in Equation 13, the error equation of motion may be deduced as Equation 16 below.

$${\dot{\tilde{x}}}_{3 j} = - {α}_{3 j}^{2} {\dot{x}}_{3 j} + \frac{{\dot{Ψ}}_{j}}{{α}_{3 j}} \qquad [ Equation 16 ]$$

The overall composition of the observers can also be integrated, with the perturbation observer and the sliding state observer integrated in one, to return only x₁ⱼ, and it is possible to compose the control system without attaching additional sensors to the system. That is, in the sliding state observer, by adding the \dot{ψ}ⱼ term to \hat{x}₂ⱼ in consideration of the effect of perturbation, the errors in the estimated state variables caused by the effect of system uncertainty, load changes, etc., can be minimized, and by obtaining only through a sensor, there is no need to include additional sensors.

Summarizing the relations described above, the overall structure of the perturbation observer may be expressed by Equation 17 as follows.

[Equation 17]

{circumflex over (\dot{x}₁ⱼ=\hat{x}₂ⱼ−k₁ⱼsat( x̅₁ⱼ)−α₁ⱼ\tilde{x}₁j

{circumflex over (\dot{x}₂ⱼ=α₃ūⱼ−k₂ⱼsat(\tilde{x}₁ⱼ)−α₂ⱼ\tilde{x}₁ⱼ−s₀ⱼ+\hat{ψ}ⱼ

{circumflex over (\dot{x}₃ⱼ=α₃j²(−\hat{x}₃ⱼ+α₃ⱼ\hat{x}₂ⱼ+ūⱼ)

Here, \hat{ψ}ⱼ is defined as in Equation 18, and as a result of the above calculations, the perturbation can be estimated.

[Equation 18]

\ddot{Ψ}ⱼ=α₃ⱼ(−\hat{x}₃ⱼ+α₃ⱼ\hat{x}₂ⱼ)

As described above, in a controller unit 110 using an SMCSPO algorithm according to this embodiment, an observer that predicts the current state of the sliding mode controller may be added to the sliding mode control, so as to monitor and predict the actual movement of the system in consideration of the state of the system (i.e. one or more of an angle, angular velocity, current angle input, angular velocity state input, etc., obtained via an encoder signal) and sliding control gain, etc.

Furthermore, in addition to observing and predicting the movement of the system through a sliding state observer, a perturbation observer for the perturbation in the sliding mode control may be added, to estimate the perturbation, which is defined as the non-linear elements of the system, the uncertainty element of the control gain, and disturbance. In the perturbation observer, when the state

$${x}_{3} = {α}_{3} {x}_{2} - \frac{Ψ}{{α}_{3}}$$

is defined, then {circumflex over (\dot{x}₃=α₃²(−\hat{x}₃+α₃\hat{x}₂+ū) may be expressed by way of control theory and the overall structure of the perturbation observer. Thus, the state value may be estimated from the value of the sliding observer obtained beforehand and the current system input u value, and ψ may be calculated in reverse.

As such, the perturbation value of the perturbation observer can also be estimated by merely adding an arbitrarily designed state value x₃ to the observed state of the system, in other words, can be estimated from just the information according to the encoder system and the input value of the current system.

When a controller according to this embodiment is applied to a surgical robot instrument, the perturbation term can be approximated by determining x₃ and the design variables of the controller from the angle and angular velocity of the encoder, especially for those cases in which the instrument holds an object or bumps into a wall. When defining perturbation as a sum of the error due to the non-linearity of the system, the error due to the uncertainty of control gain, and the disturbance due to external loads, since the main element of the perturbation is disturbance (external loads), the perturbation estimated by the perturbation observer can be estimated as a load applied on the effector of the surgical robot instrument.

The external force measurement method for an effector as described above can also be implemented in the form of a software program, etc. The code and code segments forming the program can readily be inferred by a computer programmer in the relevant field of art. Also, the program may be stored in a computer-readable information storage medium, which may be read by a computer and executed to implement the method described above. The information storage medium may include magnetic recorded media, optical recorded media, carrier wave media, etc.

According to an embodiment of the invention as set forth above, information regarding the operational force of the instrument can be measured by an indirect method.

Also, the information on the operational force of the instrument can be obtained by an indirect method to be utilized in implementing a technology for a realistic sensory device.

By implementing such a technology for a realistic sensory device, it is possible to perform surgery more safely.

Also, by measuring the operational force of the instrument and adjusting the strength accordingly, it is possible to avoid damaging a patient's internal organ while holding the organ during surgery, and hence to conduct surgery safely.

Furthermore, whereas a regular motor may perform position control, applying a torque control technique for adjusting and controlling the driving force of a motor may involve using the operational force as an input signal, and the operational force obtained according to an embodiment of the invention can hence be used as an input signal during the torque control (force control) of the motor.

While the present invention has been described with reference to particular embodiments, it is to be appreciated that various changes and modifications can be made by those skilled in the art without departing from the spirit and scope of the present invention as defined by the appended claims.

1. A surgical robot system comprising:
a driving motor unit configured to generate and output an encoder signal corresponding to state information of a system; and
a controller unit configured to receive the encoder signal as input and compute an external force applied on an instrument using an SMCSPO (sliding mode control with sliding perturbation observer) algorithm,
wherein the controller unit comprises:
a sliding state observer configured to estimate a state variable for computing a perturbation value by using the state information of the system, the state information including a rotation angle and an angular velocity of a motor within the driving motor unit; and
a perturbation observer configured to compute the perturbation value by using the estimated state variable and using an equation \hat{ψ}ⱼ=α₃ⱼ(−\hat{x}₃ⱼ+α₃ⱼ\hat{x}₂ⱼ), where \hat{ψ}ⱼ is the perturbation value, and α₃ⱼ is gain, and wherein the perturbation value is to be computed as the external force.

2. The surgical robot system according to claim 1, wherein the encoder signal includes information regarding one or more of a rotation angle of a motor and a rotation angular velocity of a motor.

3. A method of measuring an external force applied on an effector of a surgical robot system comprising a driving motor unit, an instrument, and a controller unit, the method performed by the controller unit, the method comprising:
receiving an encoder signal from the driving motor unit as input, the encoder signal corresponding to state information of a system; and
computing the external force applied on the effector by using the inputted encoder signal and an SMCSPO (sliding mode control with sliding perturbation observer) algorithm,
wherein computing the external force comprises:
estimating a state variable for computing a perturbation value by using the state information of the system, the state information including a rotation angle and an angular velocity of a motor within the driving motor unit; and
computing the perturbation value by using the estimated state variable and using an equation \hat{ψ}ⱼ=α₃ⱼ(−\hat{x}₃ⱼ+α₃ⱼ\hat{x}₂ⱼ), where \hat{ψ}ⱼ is the perturbation value, and α₃ⱼ is gain, and wherein the perturbation value is to be computed as the external force.

4. The method according to claim 3, wherein the encoder signal includes information regarding one or more of a rotation angle of a motor and a rotation angular velocity of a motor.

5. A recorded medium readable by a digital processing device, tangibly embodying a program of instructions executable by the digital processing device for performing the method disclosed in claim 3.

6. A recorded medium readable by a digital processing device, tangibly embodying a program of instructions executable by the digital processing device for performing the method disclosed in claim 4.
